(19) United States
(12) Patent Application Publication
(10) Pub. No.: US 2008/0090235 A1
(43) Pub. Date: Apr. 17, 2008

(54) Unstructured nucleic acid PCR primers and methods of using the same

(76) Inventors: Zohar Yakhini, Ramat Hasharon (IL); Doron Lipson, Rehovot (IL); Jeffrey R. Sampson, San Francisco, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/580,773

(22) Filed: Oct. 13, 2006

(51) Int. Cl. C12Q 1/68; C12P 19/34

(52) U.S. Cl. 435/6; 435/91.2

(57) ABSTRACT

A polymerase chain reaction (PCR) mixture containing at least one unstructured nucleic acid primer pair is provided. In certain embodiments, the mixture may also contain: nucleotides, a DNA polymerase, and PCR reaction reagents, as well as a nucleic acid sample. The reaction mixture may be employed in, for example, a PCR reaction.

### BACKGROUND

PCR methods are core to a variety of diagnostic methods, e.g., high-throughput SNP genotyping, and serve as a foundation for applications in forensic analysis, including human identification and paternity testing, the diagnosis of infectious diseases, whole-genome sequencing, and pharmacogenomic studies aimed at understanding the connection between individual genetic traits, drug response and disease susceptibility.

The efficiency of many PCR methods, particularly those that employ multiplex PCR methods in which several different products are amplified in a single reaction, is often low because primers hybridize to each other, rather than to the template to be amplified.

### SUMMARY

A polymerase chain reaction (PCR) mixture containing at least one unstructured nucleic acid primer pair is provided. In certain embodiments, the mixture may also contain: nucleotides, a DNA polymerase, and PCR reaction reagents, as well as a nucleic acid sample. The reaction mixture may be employed in, for example, a PCR reaction.

In certain embodiments, the PCR mixture may be a multiplex PCR reaction mixture containing at least two different unstructured nucleic acid primer pairs.

In one embodiment, employment of an unstructured nucleic acid primer pair, i.e., a pair of primers containing so-called “unstructured nucleic acid”, in an amplification reaction reduces the amount of dimer formation between the primers of the PCR reaction mixture, as compared to an otherwise identical multiplex PCR reaction mixture in which primers containing only natural bases are employed. As such, PCR methods that employ the subject PCR mixture are, in certain cases, more efficient at producing amplification products than an equivalent PCR reaction mixture that contains primers made from only naturally-occurring residues.

In one embodiment, a greater number of different amplification products can be produced using multiplex PCR reaction mixture containing UNA primers, as compared to an otherwise identical multiplex PCR reaction mixture in which primers containing only natural residues are employed. For example, a subject reaction mixture can be used to amplify sequences from a larger number of different regions in a genome of interest than an otherwise identical reaction mixture that contains primers containing only natural nucleotide residues.

### BRIEF DESCRIPTION OF THE FIGURES

FIG. 1 shows the chemical structures of several UNA nucleotides that may be used in making unstructured nucleic acid primers.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “assessing” includes any form of measurement, and includes determining if an element is present or not. The terms “determining”, “measuring”, “evaluating”, “assessing” and “assaying” are used interchangeably and includes quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “nucleoside” and “nucleotide” are intended to include those moieties that contain not only the known purine and pyrimidine base moieties, but also other heterocyclic base moieties that have been modified. Such modifications include methylated purines or pyriridines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “deoxyribonucleic acid” and “DNA” as used herein mean a polymer composed of deoxyribonucleotides.

Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T, G, and C of one sequence is then aligned with a T, A, C, and G, respectively, of the other sequence. Thus, two sequences need not have perfect homology to be “complementary” under the invention, and in most situations two sequences are sufficiently complementary when at least about 85% (preferably at least about 90%, and most preferably at least about 95%) of the nucleotides share base pair organization over a defined length of the molecule.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solutio. In other words, a mixture is not addressable. To be specific, an array of surface-bound polynucleotides, as is commonly known in the art and described below, is not a mixture of surface-bound polynucleotides because the species of surface-bound polynucleotides are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

An “oligonucleotide” is a nucleotide multimer of about 2 to about 200 nucleotides in length (e.g., about 10 to about 100 nucleotides or about 30 to about 80 nucleotides) while a “polynucleotide” or “nucleic acid” includes a nucleotide multimer having any number of nucleotides. Oligonucleotides may be synthetic or enzymatically produced.

A “primer” is an oligonucleotide can be extended from its 3′ end by the action of a polymerase. An oligonucleotide that cannot be extended from it 3′ end by the action of a polymerase is not a primer.

A “polymerase chain reaction” or “PCR” is an enzymatic reaction in which a specific template DNA is amplified using a pair of sequence specific primers.

A “multiplex polymerase chain reaction” or “multiplex PCR” is an enzymatic reaction in which two or more DNA fragments are co-amplified in a single reaction using a corresponding number of sequence-specific primer pairs.

The term “unstructured nucleic acid” or “UNA” for short, as will be described in greater detail below, is a nucleic acid that contains one or more UNA nucleotides that bind to naturally-occurring nucleotide with higher stability than it binds to other UNA nucleotides. In certain cases, the binding between the nucleotides of a base pair containing a UNA nucleotide and a corresponding naturally occurring nucleotide may be stronger than the binding between the nucleotides of a base pair containing only naturally occurring nucleotides. For example, an unstructured nucleic acid may contain an A′ residue and a T′ residue, where those residues correspond to non-naturally occurring forms, i.e., are analogs, of A and T. The A′ and T′ residues base pair with each other with reduced stability, as compared to their ability to base pair with naturally occurring T and A residues, respectively. UNA primers bind with a higher affinity to a complementary sequence containing naturally-occurring nucleic acid than to a complementary sequence containing unstructured nucleic acid.

An “unstructured nucleic acid primer” or “UNA primer” for short, as will be described in much greater detail below, is a primer that contains unstructured nucleic acid, as defined above. In other words, UNA primers contain nucleic acid that contains one or more UNA nucleotides that bind to naturally-occurring nucleotides with higher stability than it binds other UNA nucleotides. (Zohar-this is correct)

A primer that is made of “naturally occurring” nucleotides is a primer that is made up of naturally-occurring adenine (A), thymine (T), guanine (G), and cytosine (C) residues.

### DETAILED DESCRIPTION

A polymerase chain reaction (PCR) mixture containing at least one unstructured nucleic acid primer pair is provided. In certain embodiments, the mixture may also contain: nucleotides, a DNA polymerase, and PCR reaction reagents, as well as a nucleic acid sample. The reaction mixture may be employed in, for example, a PCR reaction. In certain embodiments, the PCR mixture may be a multiplex PCR reaction mixture containing at least two different unstructured nucleic acid primer pairs.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Certain embodiments of the subject PCR reaction mixture are described in greater detail below, followed by a description of exemplary methods in which the subject PCR reaction mixture find use. Finally, kits for performing the subject method are described.

### PCR Reaction Mixtures

As noted above, a polymerase chain reaction (PCR) reaction mixture containing an unstructured nucleic acid primer pair is provided. In certain aspects, the subject PCR reaction mixture may contain a plurality of (i.e., at least two) unstructured nucleic acid primer pairs. The PCR reaction mixture may also contain nucleotides, e.g., dGTP, dATP, dTTP and dCTP, a DNA polymerase, e.g., a thermostable DNA polymerase, and PCR reaction reagents, which may be a pH buffered solution containing salt, e.g., MgCl₂ and other components necessary for PCR. In certain embodiments, the PCR reaction mixture may further contain a nucleic acid sample. In certain embodiments, the components of the subject PCR reaction may be at a concentration suitable for PCR.

As noted above, the primers in the reaction mixture contain unstructured nucleic acid. Primers that contain unstructured nucleic acid are primers that contain one or more non-natural nucleotides (i.e., A′, G′, C′or T′; e.g., A′ and T′ and/or C′ and G′) and: a) maintain an ability to hybridize to a nucleic acid that has a complementary sequence of naturally occurring nucleotides (i.e., adenine, thymine, guanine, and cytosine) and b) exhibit a reduced ability to base-pair with primers made of unstructured nucleic acid. Primers made of unstructured nucleic acid may be referred to as “UNA primers” herein.

UNA primers have a reduced ability to base-pair with each other because of their reduced ability to form inter-molecular hydrogen bond base pairs. In a pair of UNA primers, at least one pair of complementary nucleotides (e.g., one or more of the A and T residues and/or one or more of the G and C residues) is substituted with a UNA nucleotide so that a base pair between those nucleotides is no longer formed or is formed at a reduced level. In some embodiments, at least one hydrogen bond is maintained in a modified base pair (e.g., an A′/T′ base pair), however, in certain modified base pairs, (e.g., a C′/G′ base pair) up to two hydrogen bonds may be maintained.

The melting temperature of two primers containing a single UNA base pair (e.g., a A′-T′ base pair) is approximately 2.5° C. lower than the melting temperature of the same primers containing naturally occurring nucleotides (e.g., A and T). The melting temperature of a duplex between a primer containing a single A′ UNA nucleotide and a complementary nucleic acid containing naturally-occurring nucleotides (e.g., a duplex having an A′-T base pair) is approximately 0.9° C. higher than that of an otherwise identical duplex that contains an A-T base pair instead of the A′-T base pair, and the melting temperature of a duplex between a primer containing a single T′ UNA nucleotide and a complementary nucleic acid containing naturally-occurring nucleotides (e.g., a duplex having an A-T′ base pair) is approximately 0.5° C. higher than that of an otherwise identical duplex that contains an A-T base pair instead of the A-T′ base pair. As such, depending on the sequence of the primers and the number of UNA nucleotides present in the primer, two complementary UNA primers have a Tₘ that at is least 1° C., at least 2° C., at least 4° C., at least 6° C., at least 8° C. or at least 10° C. or more lower than equivalent primers containing only naturally-occurring nucleotides. UNA primers anneal to complementary nucleic acid containing naturally occurring nucleotides with a Tₘ that is at least 1° C., at least 2° C., at least 4° C., at least 6° C., at least 8° C. or at least 10° C. higher than then Tm of two complementary UNA primers.

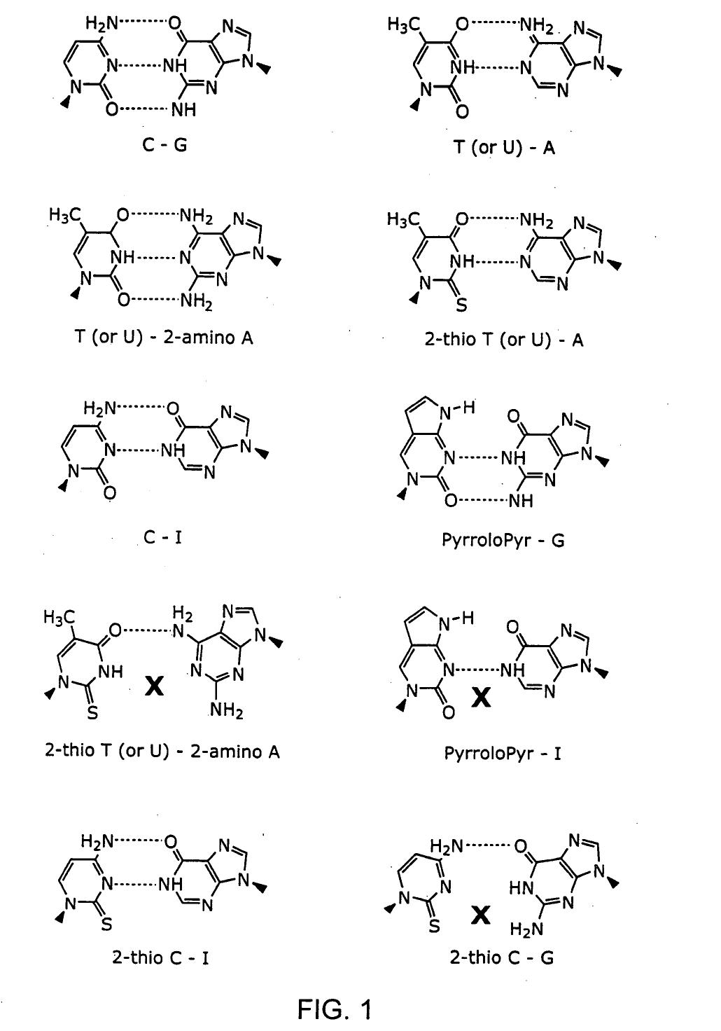

A wide variety of UNA nucleotides may be employed in a subject UNA primer. In certain embodiments, the nucleotide analogs 2,6-diaminopurine, 2-aminoadenosine, 2-thiothymidine, inosine (I), and pyrrolo-pyrimidine (P) may be used to produce UNA primers that are unable to form stable inter-molecular base pairs, yet retain their ability to form Watson-Crick base pairs with the four natural nucleotides. 2-aminoadenosine and 2-thiothymidine, for example, are unable to base pair together but are capable of base pairing with natural thymidine and natural adenine, respectively. Further, inosine and pyrroloyrimidine are unable to base pair together but are capable of binding with natural cystosine and guanine, respectively. FIG. 1 shows various exemplary UNA nucleotides base pairing with other UNA and natural nucleotides, wherein “X” denotes a base pair with low stability.

A subject primer pair may contain both UNA nucleotides and naturally occurring nucleotides, or may be entirely made up of UNA nucleotides. In particular embodiments the A and T residues of a subject primer may contain UNA nucleotides, e.g., 2,6-diaminopurine and 2-thiothmidine, respectively. In other embodiments, the G and C residues of a subject primer pair may contain UNA nucleotides e.g., inosine and/or pyrroloyrimidine, respectively. In certain cases, all of the residues of each of the primers may contain UNA nucleotides. The subject oligonucleotides may contain 1 or more, 2 or more, about 4 or more, about 6 or more, about 8 or more, about 10 or more, about 12 or more, about 16 or more or about 20 or more, up to about 24 or 30 or more, UNA nucleotides. In certain embodiments, the UNA nucleotides may be positioned towards the 3′ end of the primer, e.g., at the 3′ terminal nucleotide, for example.

Further description of UNAs may be found in published U.S. patent applications 20030211474, 20040086880, and Kutyavin et al., (Nucl. Acids. Res. 2002 30:4952-4959) which are incorporated by reference in their entirety. As detailed therein, UNAs may be made enzymatically or synthetically. Exemplary UNA nucleotides are shown in FIG. 1.

The primers of an unstructured nucleic acid primer may be about 2 to about 200 bases in length. In certain embodiments, the primers may be about 10 to about 100 bases, about 12 to about 80 bases, about 15 to about 60 bases, or about 20 to about 40 bases in length. In particular embodiments, a UNA primer may be about 16 to about 30 bases in length.

A subject PCR reaction mixture may contain an UNA primer pair, where a primer pair contains two primers of a specific sequence that can be employed in a polymerase chain reaction to amplify a product from a template. In certain embodiments, the subject PCR reaction mixture may be a multiplex PCR reaction mixture containing at least two (i.e., a plurality) of UNA primer pairs (e.g., two or more, e.g., 3 or more, 4 or more, 5 or more, 6 or more, 7 or more, 8 or more, 9 or more, or 10 or more UNA primer pairs, up to about 20, 30, 40, 50, 60, 70, 80 or 100 or more UNA primer pairs) that bind to target nucleic acids and produce a corresponding number of amplification products if the target nucleic acids are also present in the reaction. In certain cases the amplification products are all of different lengths and may be non-overlapping and distinct (e.g., do not share a common nucleotide sequence).

In one multiplex embodiment, the primer pairs may bind to and amplify products from different regions of a genome under analysis. For example, each primer pair of the plurality of primer pairs may amplify a product from a different chromosome of the genome under analysis. In certain embodiments, the primer pairs may bind to and amplify a single copy locus of the genome under analysis, i.e., a unique sequence that is represented once per haploid genome. In certain cases, the primer pairs may bind to and amplify products if certain single nucleotide polymorphisms (SNPs) are present. In another embodiment, the primer pairs amplify SNP-containing regions.

In another multiplex embodiment (and as will be described in greater detail below) the primer pairs bay bind to and amplify the cDNA copies of the mRNA transcripts of a plurality of different genes, or in multiplex ligation-dependent probe mplification (MLPA) or MAPH (multiplex amplification and probe hybridization) methods.

In certain embodiments, one or both of the primers in each primer pair may be detectably labeled (e.g., with a fluorescent or mass-labeled). Labels of interest include directly detectable and indirectly detectable non-radioactive labels such as fluorescent dyes. Fluorescent labels that find use in the subject invention include a fluorophore moiety. Specific fluorescent dyes of interest include: xanthene dyes, e.g. fluorescein and rhodamine dyes, such as fluorescein isothiocyanate (FITC), 6-carboxyfluorescein (commonly known by the abbreviations FAM and F),6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TANRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, etc. Suitable labels for mass tagging are found in published U.S. patent applications 20060003352 and 20050239086. In certain cases, the primers may be “mass-labeled” by the use of modified bases.

The primers of the reaction mixture may be designed to have similar thermodynamic properties, e.g., similar Tms, G/C content, hairpin stability, and in certain embodiments may all be of a similar length, e.g., from 18 to 30 nt, e.g., 20 to 25 nt in length, etc. The primer may be employed at the same concentration as each other, or at different concentrations.

The amount of primer present in a subject reaction mixture may vary greatly. In certain embodiments, each primer pair may be present at an amount in the range of 1 pM to 100 pM, e.g., 3 pM to 50 pM, although primer concentrations well outside of these ranges may be employed. In a 50 μl reaction these amounts may correspond to concentrations of 0.02 μM to 2 μM, e.g. 0.06 μM to 1 μM.

In addition the UNA primers, exemplary reaction buffers and DNA polymerases that may be employed in the subject reaction mixture include those described in, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y. Reaction buffers and DNA polymerases suitable for PCR may be purchased from a variety of suppliers, e.g., Invitrogen (Carlsbad, Calif.), Qiagen (Valencia, Calif.) and Stratagene (La Jolla, Calif.). Exemplary polymerases include Taq, Pfu, Pwo, UlTma and Vent, although many other polymerases may be employed in certain embodiments. Guidance for the reaction components suitable for use with a polymerase as well as suitable conditions for its use, is found in the literature supplied with the polymerase.

In certain embodiments, a subject reaction mix may further contain a nucleic acid sample. The nucleic acid template in the subject reaction mix may contain genomic DNA or an amplified version thereof (e.g., genomic DNA amplified using the methods of Lage et al, Genome Res. 2003 13:294-307 or published patent application US20040241658, for example), cDNA, or DNA from a pathogen-infected subject, for example. In exemplary embodiments, the nucleic acid sample may contain genomic DNA from a mammalian cell such a human, mouse, rat or monkey cell. If present, nucleic acid in the nucleic acid sample may be at a concentration of about 0.1 pg/μl to about 1 pg/μl, about 1 pg/μl to about 10 pg/μl, 10 pg/μl to about 0.1 ng/μl, 0.1 ng/μl to about 1 ng/μl, about 1 ng/μl to about 10 ng/μl, about 10 ng/μl to about 100 ng/μl, about 100 ng/μl to about 1 μg/μl, although concentration outside of these ranges are readily employed. Since, as will be described below, the subject reaction mixture may be employed for diagnostic purposes, the nucleic acid of the nucleic acid sample may or may contain target nucleic acid for all of the UNA primer pairs in the reaction mix.

The above-described reaction mixture may be employed in multiplex PCR methods to co-amplify 10 or more products, 15 or more or products, 20 or more products, 25 or more products, 30 or more products, up to about 50 products or up to about 100 or more products, in certain cases without significant formation of primer dimers. The subject multiplex PCR methods may be employed to amplify at least 1.5 times, at least 2 times, at least 3 times, at least 5 times or at least 10 times the number of target PCR products than an otherwise identical methods that employ primers containing only naturally-occurring nucleotides.

As would be readily apparent, the above-described UNA primer pairs may be designed using any one of a number of different programs specifically designed to design primer pairs for multiplex PCR methods. For example, the primer pairs may be designed using the methods of Yamada et al. (PrimerStation: a highly specific multiplex genomic PCR primer design server for the human genome. Nucleic Acids Res. 2006 34:W665-9), Lee et al. (MultiPrimer: software for multiplex primer design. Appl. Bioinformatics 2006 5:99-109), Vallone et al. (AutoDimer: a screening tool for primer-dimer and hairpin structures. Biotechniques. 2004 37:226-31), Rachlin et al. (Computational tradeoffs in multiplex PCR assay design for SNP genotyping BMC Genomics. 2005 6:102) or Gorelenkov et al. (Set of novel•tools for PCR primer design Biotechniques. 2001 31:1326-30). In one embodiment, methods using optimization approaches for graph theory methods may be employed. In these methods the task of designing an optimal primer set for multiplex PCR is translated into a graph theory problem. Nodes represent the different molecules to be amplified (such as genomic loci) and edges represent potential conflicts, including primer-dimer potential. An efficient coloring of such a graph represents an efficient multiplexing scheme for the original set of loci. Such methods are described in Lipson (Master's Thesis entitled “Optimization Problems in Design of Oligonucleotides for Hybridization-based Methods”, Technion-Israel Institute of Technology, 2002), which is incorporated by reference in its entirety. In a particular embodiment, a plurality of primer pairs may be designed using a program, and nucleotide residues in the designed primer sequence may substituted for appropriate UNA nucleotides.

### Method of Sample Analysis

A method assessing a genomic sample is also provided. In general terms, this method includes: a) combining the above-described PCR reaction mixture with a nucleic acid sample; b) maintaining the PCR reaction mixture and nucleic acid sample under conditions suitable for PCR; and c) assessing the amplification products produced by the PCR. In certain embodiments, the presence and/or abundance of each amplification product may be assessed to provide an evaluation of the sample.

In certain embodiments, results obtained from a subject assay may be compared to control results to provide an evaluation of the nucleic acid sample. The control results may be obtained using a control nucleic acid sample, e.g., a sample known to contain binding sites for the primer pairs in the reaction.

PCR conditions of interest include those well known in the art (e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y. for example). The amounts of the amplification products may be assessed after any number of rounds of PCR amplification (i.e., successive cycles of denaturation, re-naturation and polymerization). In certain embodiments, the amount of any amplification product may be assessed a stage at which the nucleic acid amplification occurs linearly (i.e., during the linear phase of the amplification reaction) or after the reaction rate has reached a plateau. In certain embodiments the amounts of each amplification product may be assessed after 12 and before 40 successive rounds of amplification, e.g., 12 to 16 rounds, 16 to 20 rounds, 20 to 24 rounds, 24 to 30 rounds, or 30 to 40 rounds of amplification. In general, the number of rounds of application employed provides an amount of amplification product that is detectable using the detection system employed. The optimal number of rounds of amplification employed in the subject methods may vary according to the primer set used, as discussed above. The optimal number of rounds of amplification for each sample is readily determinable. In certain embodiments, the amount of the primers employed in the PCR reactions is limiting.

After amplification, the amplification products may be detected. The amount and/or presence of amplification products may be detected by any suitable means, including, but not limited to: separating the products according to their size using a separation device (for example, a column, gel or filter) and independently detecting each of the separated products by, e.g., a) contacting the separated products with a detectable (e.g., fluorescent) DNA binding agent and assessing the amount of bound agent, b) by detecting absorbance at 260 nm, or, c) detecting the presence of a detectable label if a detectably labeled primer was employed in the amplification reaction. The methods described above are readily automated. In certain embodiments, a microfluidic system may be employed for analysis of amplification products. One representative system that may be employed is a microcapillary device such as the DNA 7500 LabChip and Bioanalyzer of Agilent Technologies (Palo Alto, Calif.).

When employed in polymerase chain reaction with target nucleic acids for the primer pairs, each UNA primer pair of a subject multiplex PCR mixture may be expected to produce an amplification product of a length that may be within the range of 50 bp to 5 kb, e.g., 50 bp to 3 kb, 60 bp to 2 kb, 100 bp to 1 kb or 1 kb to 3 kb, although UNA primer pairs that produce amplification products outside of this length range may be employed in certain embodiments. Collectively, the plurality of UNA primers in the multiplex reaction mixture may produce a corresponding plurality of amplification products if target nucleic acid for those primer pairs is present in the reaction mix.

In certain embodiments, the amplification products may be physically resolvable by size. In certain embodiments, the amplification products may have different lengths, and may be distributed across a size range. In certain embodiments, the size of the amplification products may be distributed across a size range that is between 50 bp to 5 kb in size, although in certain embodiments a wider or narrower range may be employed. In one embodiment, the primer pairs produce a size ladder of amplification products that are distributed between 50 bp and 5 kb bp in length, 50 bp and 1 kb in length, or 100 bp and 5 kb in length. Depending on the range of length of the amplification products and the number of primer pairs employed, the length difference between any two amplification products may be at least 50 bp, at last 100 bp or at least 200 bp, for example. In one embodiment, the plurality of UNA primer pairs may produce a ladder of amplification products, where the size difference between consecutive amplification products is about 5 bp to 50 bp, 50 bp to 150 bp, e.g., about 80 bp to 120 bp, or 150 bp to 250 bp, e.g., 180 bp to 200 bp, in length.

If a subject multiplex PCR mixture is employed in a PCR method, the method may produce at least 10, at least 15, at least 20, at least 25, at least 30, at least 35, at least 40 or at least 50 or more resolvable products.

The results obtained from an assay may be graphed, and, in certain embodiments, the sizes and/or the abundance of the amplification products may be calculated. Any evaluation may be qualitative or quantitative.

The amplification methods may be performed using a thermocycler, e.g., a thermocycler from Perkin Elmer Wellesley, MA, Biorad (Hercules, Calif.) or Stratagene (La Jolla, Calif.) or another manufacturer. As such, a thermocycler containing the subject PCR mixture is also provided.

Kits

Kits for use in accordance with the subject methods are also provided. The kits at least, as described above, a UNA primer pair for producing an amplification products of a particular size. In particular embodiments, the kit may contain a plurality of UNA primer pairs for producing a corresponding plurality of amplification products of a range of different sizes.

A kit may include one or more of: a nucleic acid sample that contains binding sites for the primer pairs, a polymerase, e.g., a thermostable polymerase, or reaction buffer components for performing PCR, e.g., MgCl₂ and nucleotides, etc.

A subject kit may further include one or more additional components necessary for carrying out an array-based genome assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay. Such kits also typically include instructions for use in practicing array-based assays.

The kits may also include a computer readable medium including and instructions that may include directions for use of the invention.

The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

Utility

The subject PCR reaction mixture may be employed in any PCR method to provide quantitative results (e.g., to assess the abundance of a nucleic acid in a sample), quantitative results (e.g., to determine if a particular nucleic acid is present in a sample) or for cloning purposes (e.g., to ligate to nucleic acids together). For example, the instant mixture may be employed to: a) quantitatively assess the abundance of RNA molecules in an RNA sample (using, e.g., RT-PCR methods), or the copy number of regions of a genome, e.g., to determine if a genome contains a deleted or duplicated region, relative to another genome; b) qualitatively assess the presence of a particular nucleic acid in a sample, e.g., in diagnostic or mutation detection methods; or c) in other methods, e.g., in methods in which two or more nucleic acids may be amplified and ligated together. In certain embodiments, the subject PCR reaction mixture may be employed in AFLP, RFLP, MLPA (Multiplex Ligation-dependent Probe Amplification) and MAPH (multiplex amplification and probe hybridization) methods. Such methods are reviewed by Sellner et al (Hum. Mutat. 2004 23:413-9). Rooms et al. (Hum. Mutat. 2005 25:513-24) and Schouten et al (Nucl.Acids Res. 2002 30:e57) which are incorporated by reference herein for the description of those methods.

The above described PCR reaction mixture and sample analysis methods finds use in a variety of diagnostic, research and clinical applications, including detecting infectious microorganisms, whole-genome sequencing, forensic analysis, and high throughput genotyping. For example, the multiplex PCR reaction mixture and sample analysis methods find use in diseases diagnosis (see, e.g., Elnifro, et al. Clinical Microbiology Reviews, 13:559 (2000)), paternity testing (see, e.g., Hidding and Schmitt, Forensic Sci. Int., 113:47 (2000); Bauer et al., Int. J. Legal Med. 116:39 (2002)), preimplantation genetic diagnosis (see, e.g., Ouhibi, et al., Curr Womens Health Rep. 1: 138 (2001)), microbial analysis in environmental and food samples (see, e.g., Rudi et al., Int J Food Microbiology, 78:171 (2002)), and veterinary medicine (see, e.g., Zarlenga and Higgins, Vet Parasitol. 101:215 (2001)), among others.

The subject multiplex PCR reaction mix may also be used to investigate entire genomes or sub-regions thereof, particularly for sequence variations, e.g., single nucleotide polymorphisms, or SNPs. For example, multiplex PCR has been used in the analysis of the relationship between genetic variation and phenotype by making use of polymorphic DNA markers. Common SNPs occur at an average frequency of more than 1 per kilobase in human genomic DNA. Some SNPs, particularly those in and around coding sequences, are the direct cause of therapeutically relevant phenotypic variants.

The subject multiplex PCR reaction mix may be employed to investigate a number of clinically important polymorphisms, for example, the apoE2/3/4 variants are associated with different relative risk of Alzheimer's and other diseases (see Cordor, et al., Science 261(1993), and the SNPs associated with cystic fibrosis (see Mutat Res. 2005 573:195-204), as well as many cancers, diabetes, heart disease, hypercholesterolemia and inflammatory diseases, as well a number of hereditary diseases.

In one embodiment, the subject multiplex PCR methods may be used to amplify a plurality of different regions from a test nucleic acid sample. The amplified regions may be subsequently analyzed for SNPs by methods that rely on, e.g., primer extension, primer ligation, sequencing, electrophoresis, hybridization or mass spectrometry, etc. In other embodiments, the primers themselves are designed so that they can only be extended if a certain SNP is present. In this case, the profile of the amplification products (i.e., which indicates the presence or absence of each amplification product) may indicate the genotype of the sample.

In other embodiments, the subject multiplex PCR reaction mixture may be employed to evaluate the abundance of a plurality of different RNA molecules in a sample. In these embodiments, a sample containing RNA, e.g., mRNA, is subjected to reverse transcriptase conditions to produce cDNA, and regions of the cDNA are amplified using a subject multiplex PCR reaction mixture. Such methods, termed “reverse transcriptase-polymerase chain reaction” or “RT-PCR” methods are generally well known in the art (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.).

In another embodiment, the amplification products may be labeled and hybridized to a polynucleotide array containing surface bound polynucleotides that bind to those products. The level of binding of the labeled amplification products to the array indicates the amount of the amplification products in the sample.

All statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A polymerase chain reaction (POR) mixture comprising:
a) a pair of unstructured nucleic acid (UNA) primers, wherein said pair comprises a first primer comprising a first UNA nucleotide and a second primer comprising a different UNA nucleotide and wherein said first UNA nucleotide and said different UNA nucleotide base pair with naturally occurring nucleotides that are complementary to each other;
b) nucleotides;
c) a DNA polymerase; and
d) POR reaction reagents.

2. The POR mixture of claim 1, wherein said mixture further comprises a nucleic acid sample.

3. The POR mixture of claim 2, wherein said nucleic acid sample comprises genomic DNA.

4. The PCR mixture of claim 2, wherein said nucleic acid sample comprises binding sites for said first primer and said second primer.

5. The PCR mixture of claim 1, wherein one or both of said unstructured nucleic acid primers is detectably labeled.

6. The PCR mixture of claim 1, wherein said PCR mixture is a multiplex PCR mixture and contains at least two different pairs of unstructured nucleic acid primers.

7. The PCR mixture of claim 6, wherein said pairs of unstructured nucleic acid primers bind to different regions of a DNA sample.

8. The PCR mixture of claim 6, wherein said multiplex PCR mixture produces different sized products when employed in an amplification reaction.

9. The PCR mixture of claim 6, wherein said multiplex PCR mixture comprises from 5 to 50 different primer pairs.

10. The PCR mixture of claim 2, wherein said nucleic acid sample comprises cDNA.

11. The PCR reaction mixture of claim 1, wherein said polymerase is a thermostable DNA polymerase

12. (canceled)

13. A thermocycler comprising the PCR mixture of claim 1.

14. A method comprising:
a) combining a PCR reaction mixture of claim 1 with a nucleic acid sample;
b) maintaining said PCR reaction mixture under PCR conditions to produce an amplification product; and
c) size separating said amplification product, to evaluate said amplification product.

15. (canceled)

16. The method of claim 14, wherein said evaluating includes comparing said amplification product to a control amplification product.

17. The method of claim 14, wherein said nucleic acid sample comprises genomic DNA.

18. The method of claim 14, wherein said evaluating is quantitative or qualitative.

19. A kit comprising a pair of unstructured nucleic acid primers of claim 1.

20. The kit of claim 19, wherein said kit comprises at least two different pairs of unstructured nucleic acid primers.

21. The kit of claim 1 9, further comprising PCR reagents.

22. The kit of claim 19, further comprising a control nucleic acid sample comprising binding sites for said pairs of unstructured nucleic acid primers.
